(19) United States
(12) Patent Application Publication
(10) Pub. No.: US 2007/0059789 A1
(43) Pub. Date: Mar. 15, 2007

(54) Process for the enzymatic synthesis of ethers

(75) Inventors: Uwe Bornscheuer, Greifswald (DE); Burghard Gruning, Essen (DE); Geoffrcy Hills, Essen (DE); Patrick Mobius, Demmin (DE); Oliver Thum, Ratingen (DE); Christian Weitemeyer, Essen (DE)

Correspondence Address: Leopold Presser;Scully, Scott, Murphy & Presser, 400 Garden City Plaza, Garden City, NY, 11530, US

(73) Assignee: Goldschmidt GmbH, Essen (DE)

(21) Appl. No.: 11/519,183

(22) Filed: Sep. 11, 2006

(30) Foreign Application Priority Data
Sep. 14, 2005 (DE) 10 2005 043 669.2

(51) Int. Cl. C12P 1/00; C12P 7/02

(52) U.S. Cl. 435/41; 435/155

(57) ABSTRACT

An enzymatic process for preparing ethers is disclosed which includes reacting one or more alcohols with an ether in the presence of at least one enzyme.

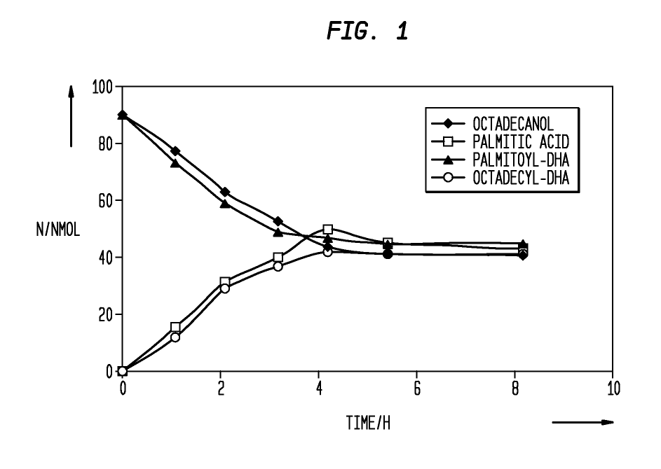

### FIELD OF THE INVENTION

The present invention relates to an enzymatic process for preparing ethers.

### BACKGROUND OF THE INVENTION

Ethers represent an important class of chemical products which in some cases are prepared in very large quantities. A number of chemical processes are known for preparing ethers. For example, and in the case of the production of methyl t-butyl ether (MTBE), addition of methanol onto isobutene with catalysis by acidic ion exchangers is known. Further processes use alkyl halides (Williamson's ether synthesis) or Lewis acids such as ZnCl₂.

It is common to all these prior processes, however, that relatively drastic conditions must be applied in order to carry out the syntheses, so that side reactions are unavoidable especially on use of sensitive raw materials (e.g., unsaturated alcohols). To make it possible to use these products in sectors where a high purity is required, e.g., in cosmetic formulations, normally elaborate additional workup and purification steps are therefore necessary.

To date, the enzymatic synthesis of ethers has not been extensively investigated. Research has concentrated instead on investigations of the microbial degradation of ethers, especially in relation to the degradation of MTBE contaminations in water and soil.

The enzymes responsible for this ether cleavage cannot be employed in the synthesis of ethers. The reason for this derives from the mechanisms of degradation, where the ether linkage is cleaved by oxygenation, oxidation by P450 enzymes, dealkylation, reduction or lyases (see G. White, N. J. Russell, E. C. Tidswell, Microbiol. Rev. 1996, 60, 216-232). All of these mechanisms for the degradation of ethers are irreversible and cannot be utilized for synthesis. The enzyme referred to as β-etherase is also unsuitable for ether synthesis for mechanistic reasons (see E. Masai, Y. Katayama, S. Kubota, S. Kawai, M. Yamasaki, N. Morohoshi, FEBS Letters 1993, 323, 135-140).

A single process for the enzymatic synthesis of an ether has been disclosed to date. This entails a 1-acyldihydroxyacetone phosphate (e.g., 1-palmitoyl-DHAP) which is converted into a 1-alkyldihydroxyacetone phosphate (e.g., 1-palmityl-DHAP) using alkyldihydroxyacetone-phosphate synthase (ADAPS, EC 2.5.1.26) (cf. Scheme 1) (A. J. Brown, F. Snyder, J. Biol. Chem. 1982, 257, 8835-8839; A. J. Brown, F. Snyder, Methods Enzymol. 1992, 209, 377-384; A. Zomer, P. Michels, F. Opperdoes, Mol. Biochem. Parasitol. 1999, 104, 55-66).

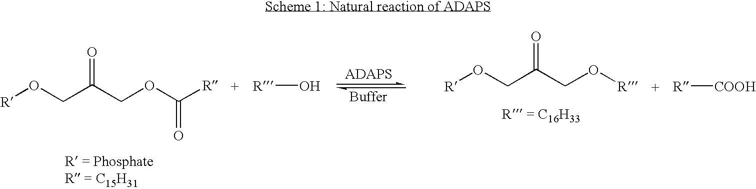

ADAPS has been isolated from various organisms, e.g., Trypanosoma sp. and Leishmania sp., and biochemically characterized. However, reports in this connection have dealt exclusively with the catalytic effect of ADAPS in its natural function and under reaction conditions similar to those of the physiological function, i.e., the known process is restricted to reactions using 1-acyldihydroxyacetone phosphates as precursors. Nothing is yet known about the suitability of this or other enzymes for industrial biocatalysis with other, more easily accessible substrates than 1-acyldihydroxyacetone phosphates. In addition, the processes described in the prior art have the disadvantage in that they each involve aqueous systems in which a large number of organic compounds are insoluble or have only limited solubility. Thus, the possible applications in industrial organic synthesis are greatly restricted.

### SUMMARY OF THE INVENTION

The present invention provides a process which makes it possible to prepare ethers with enzyme catalysis under conditions like those normally used for the industrial production of organic chemical compounds.

It has surprisingly been found that enzymes, preferably from the group of transferases that transfer alkyl groups (EC 2.5.x.y), especially alkyldihydroxyacetone-phosphate synthase (ADAPS, EC 2.5.1.26), are able to catalyze the synthesis of ethers even with use of non-natural substrates and in nonaqueous systems.

This invention makes it possible to use a large number of substrates, thus opening up a wide range of applications of the process of the invention.

The present invention therefore relates to a process for preparing ethers which involves at least one enzyme and uses non-natural substrates.

### BRIEF DESCRIPTION OF THE DRAWINGS

The sole figure of the present application illustrates the progress of an ADAPS-catalyzed reaction as in Example 4.

### DETAILED DESCRIPTION OF THE INVENTION

The present invention, which provides an enzymatic process for preparing ethers, will now be described in greater detail by referring to the following discussion. It is again observed that the present invention provides a process to prepare ethers with enzyme catalysis under conditions like those normally used for the industrial production of organic chemical compounds. As such, the inventive process represents an advancement in the field of ether synthesis.

A particular aspect of the invention is a process for preparing compounds of the general formula (I)
R—O—R¹ (I)
in which
R is a hydrocarbon radical of a substituted or unsubstituted alcohol which is optionally branched and/or comprises one or more multiple bond(s) and has 1 to 30 C atoms, preferably 2 to 30 C atoms, particularly preferably 3 to 22 C atoms, especially 8 to 18 C atoms, and which may optionally comprise additional hydroxy groups and/or hydroxy groups edierified with organic radicals,
   which comprises reacting one or more alcohols of the general formula (II)
   R—OH (II)
   in which R is as defined above, in the presence of at least one enzyme as a catalyst, with a compound of the general formula (III),
   R¹—O—R² (III)
   in which
R¹ is a linear or branched, substituted or unsubstituted alkyl or alkenyl group having 2 to 30 carbon atoms, optionally comprising additional carbonyl groups and/or hydroxy groups and/or hydroxy groups etherified with organic radicals and/or hydroxy groups esterified with organic or inorganic acids, and
R² is an acyl radical of a substituted or unsubstituted acid which is optionally branched and/or comprises one or more multiple bond(s) and/or hydroxy groups and has 2 to 30 carbon atoms,
   with the proviso that when R¹ is a 1-dihydroxyacetone phosphate, R² is not an acyl radical of a naturally occurring fatty acid having 8 to 30 carbon atoms.

It is preferred according to the invention to use a nonaqueous reaction system that in the context of this invention consists of a reaction mixture which is suitable for the desired synthesis, and which comprises less than 30% by weight, preferably less than 10% by weight, particularly preferably less than 5% by weight, of water.

Besides the reactants, the biocatalyst and further substances necessary to maintain the enzymatic activity, such as, for example, cofactors such as, for example, FADH₂/FAD, NAD(P)H/NAD(P)⁺, metal ions, stabilizers or detergents such as, for example, Triton X-100, it is possible according to the invention to use organic solvents such as, for example, pentane, hexane, heptane, octane, diethyl ether, MTBE, dioxanes, furans, diisopropyl ether, tetrahydrofuran, 2-butanol, t-butanol, methylcyclohexane, toluene, acetone, dimethyl sulfoxide, dimethylformamide, dichloromethane, chloroform. It is also possible according to the invention to use other substances as solvents, for example, compounds under supercritical conditions (e.g., supercritical carbon dioxide, supercritical propane or other hydrocarbons) or ionic liquids (e.g., EMIM-PF₆, BMIM-PF₆, BMIM-BF₄).

The use of suitable buffer systems may be necessary, for example, a buffer consisting of 50 mM potassium phosphate, pH 7.5, 50 mM NaF, 0.1% Triton X-100.

The alcohols of the general formula II, which can be used in the process of the invention, may be substituted and/or unsubstituted alcohols that are optionally branched and/or comprise one or more multiple bonds and have 1 to 30 carbon atoms, preferably 2 to 30 carbon atoms, particularly preferably 3 to 22 carbon atoms, in particular 8 to 18 carbon atoms. Illustrative examples of such alcohols include, but are not limited to, methanol, ethanol, propanol, butanol, pentanol, hexanol, octanol and their isomers such as i-propanol, i-butanol, 2-ethylhexanol, isononyl alcohol, isotridecyl alcohol, and polyhydric alcohols such as 1,6-hexanediol, 1,2-pentanediol, glycerol, diglycerol, triglycerol, polyglycerol, ethylene glycol, diethylene glycol, triethylene glycol, and polyethylene glycol.

In some embodiments of the present invention, alcohols which are prepared by known processes from monobasic fatty acids based on natural vegetable or animal oils having 6 to 30 carbon atoms, preferably 8 to 22 carbon atoms, in particular 8 to 18 carbon atoms, such as caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, petroselinic acid, elaidic acid, arachidic acid, behenic acid, erucic acid, gadoleic acid, rape oil fatty acid, soybean oil fatty acid, sunflower oil fatty acid, tallow oil fatty acid, palm oil fatty acid, palm kernel oil fatty acid, coconut fatty acid, alone or in a mixture can also be employed.

Examples of the radical R¹ in the general formula III are alkyl radicals from dihydroxyacetone, 1,2-propylene glycol, 1,3-propylene glycol, neopentyl glycol, 1,6-hexanediol-,1,2-pentanediol, glycerol, trimethylolpropane, pentaerythritol or sorbitol. It is possible according to the invention for any further hydroxy groups present to be esterified with an inorganic acid such as phosphoric acid or sulfuric acid, or an acyl radical. This is the case, for example, on use of alkyl radicals of 1(2)-monoacylglycerides, 1,2-diacylglycerides or 1-acylethylene glycol. It is also possible according to the invention for any further hydroxy groups present to be etherified with a further alcohol such as, for example, on use of alkyl radicals from diglycerol, triglycerol, polyglycerol, diethylene glycol, triethylene glycol or polyethylene glycol.

Examples of the radical R² in the general formula III are acyl radicals which are substituted or unsubstituted and/or branched or unbranched and/or comprise multiple bonds and/or comprise hydroxy groups of commercially available acids having 1 to 15, preferably 1 to 10, particularly preferably 1 to 6, carbon atoms, such as acetic acid, propanoic acid, butanoic acid, pentanoic acid, chloroacetic acid, and trifluoroacetic acid. Also suitable are substituted or unsubstituted acyl radicals of monobasic fatty acids based on natural vegetable or animal oils having 6 to 30 carbon atoms, in particular 8 to 22 carbon atoms, such as caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, petroselinic acid, elaidic acid, arachidic acid, behenic acid, erucic acid, gadoleic acid, linolenic acid, eicosapentaenoic acid, docosahexaenoic acid, and arachidonic acid, which can be employed alone or in a mixture. It should be noted in this regard that in the case where R¹ is a 1-dihydroxyacetone phosphate, R² cannot be an acyl radical of a naturally occurring fatty acid or in specific cases generally of a naturally occurring acid, in each case having 8 to 30 carbon atoms.

The enzymes which can be used according to the invention are preferably those from the group of transferases that transfer alkyl groups (EC 2.5.X.X), preferably an alkyldihydroxyacetone-phosphate synthase (EC 2.5.1.26). In some embodiments of the present invention, it is particularly preferably to use alkyldihydroxyacetone-phosphate synthases which are known in the art and are listed in Table 1 and 2 and have been at least, in part, disclosed in A. Zomer, P. Michels, F. Opperdoes, Mol. Biochem. Parasitol. 1999, 104, 55-66, or an analog, allele, derivative, a functional variant or a functional partial sequence thereof. The contents of this publication and of the amino acid sequences cited in Tables 1 and 2 are hereby expressly incorporated into the description of the present application.

TABLE 1
Organisms with ADAPS accession code from BRENDA

| Organism | Accession_Code | Domain (life) |
|---|---|---|
| Caenorhabditis elegans | O45218 | Eucaryota |
| Cavia porcellus | P97275 | Eucaryota |
| Dictyostelium discoideum | O96759 | Procaryota |
| Drosophila melanogaster | Q9V778 | Eucaryota |
| Homo sapiens | O00116 | Eucaryota |
| Trypanosoma brucei | O97157 | Eucaryota |
| Leishmania major | Q7YWB6 | Eucaryota |
| Mycobacterium tuberculosis | Q8VJN8 | Procaryota |
| Caenorhabditis elegans | O05784 | Eucaryota |
| Cavia porcellus | Q7TX86 | Eucaryota |
| Dictyostelium discoideum | Q8F3Y7 | Bacteria |
| Drosophila melanogaster | Q9WUW6 | Eucaryota |
| Homo sapiens | O45218 | Eucaryota |
| Trypanosoma brucei | P97275 | Eucaryota |
| Leishmania major | O96759 | Eucaryota |
| Mycobacterium tuberculosis | Q9V778 | Bacteria |
| Mycobacterium bovis | O00116 | Bacteria |
| Leptospira interrogans | O97157 | Bacteria |

TABLE 2
Organisms with ADAPS accession code from NCBI

| | Accession | Domain | |
|---|---|---|---|
| Organism | Code | of life | Reference |
| Sulfolobus | YP_256483 | Archaea | Chen L. et al, J. Bacteriol. 187 (14), |
| acidocaldarius DSM 639 | | | 4992-4999 (2005) |
| Sulfolobus | YP_255828 | Archaea | Chen L. et al, J. Bacteriol. 187 (14), |
| acidocaldarius DSM 639 | | | 4992-4999 (2005) |
| Sulfolobus | AAY81190 | Archaea | Chen L. et al, J. Bacteriol. 187 (14), |
| acidocaldarius DSM 639 | | | 4992-4999 (2005) |
| Sulfolobus | AAY80535 | Archaea | Chen L. et al, J. Bacteriol. 187 (14), |
| acidocaldarius DSM 639 | | | 4992-4999 (2005) |
| Caenorhabditis elegans | O45218 | Eukaryota | De Vet E. C. et al., Biochem. |
| | | | Biophys. Res. Commun. 242 (2), |
| | | | 277-281 (1998) |
| Drosophila melanogaster | Q9V778 | Eukaryota | Adams M. D. et al., Science 287 |
| | | | (5461), 2185-2195 (2000) |
| Homo sapiens | NP_003650 | Eukaryota | Wanders R. J. et al., J. Inherit. |
| | | | Metab. Dis. 17 (3), 315-318 (1994) |
| Mycobacterium bovis | CAD96821 | Bacteria | |
| AF2122/97 | | | |
| Mycobacterium bovis | CAD97128 | Bacteria | |
| AF2122/97 | | | |
| Mycobacterium | CAA17288 | Bacteria | Camus J. C. et al., Microbiology |
| tuberculosis H37Rv | | | (Reading, Engl.) 148 (PT 10), 2967-2973 (2002) |
| Mycobacterium | CAB08364 | Bacteria | Camus J. C. et al., Microbiology |
| tuberculosis H37Rv | | | (Reading, Engl.) 148 (PT 10), 2967-2973 (2002) |
| Archaeoglobus fulgidus | NP_069702 | Archaea | Klenk H. P. et al., Nature 390 |
| DSM 4304 | | | (6658), 364-370 (1997) |
| Mycobacterium bovis | NP_856779 | Bacteria | Garnier T. et al., Proc. Natl. Acad. |
| AF2122/97 | | | Sci. U.S.A. 100 (13), 7877-7882 (2003) |
| Mycobacterium bovis | NP_855924 | Bacteria | Garnier T. et al., Proc. Natl. Acad. |
| AF2122/97 | | | Sci. U.S.A. 100 (13), 7877-7882 (2003) |
| Aeropyrum pernix K1 | NP_147137 | Archaea | Kawarabayasi Y. et al., |
| | | | DANN Res. 6 (2), 83-101 (1999) |
| Trypanosoma brucei | AAX79694 | Eukaryota | |
| Aeropyrum pernix | C72721 | Archaea | |
| Dictyostelium | JE0365 | Eukaryota | De Vet E. C. et al., Biochem. |
| discoideum | | | Biophys. Res. Commun. 252 (3), |
| | | | 629-633 (1998) |
| Archaeoglobus fulgidus | D69358 | Archaea | Klenk H. P. et al., Nature 390 |
| | | | (6658), 364-370 (1997) |
| Sulfolobus solfataricus | NP_342963 | Archaea | She Q. et al., Proc. Natl. Acad. Sci. |
| P2 | | | U.S.A. 98 (14), 7835-7840 (2001) |
| Caenorhabditis elegans | AAU26108 | Eukaryota | WormBase Consortium, Science |
| | | | 282 (5396), 2012-2018 (1998) |
| Mycobacterium | NP_217623 | Bacteria | Camus J. C. et al., Microbiology |
| tuberculosis H37Rv | | | (Reading, Engl.) 148 (PT 10), 2967-2973 (2002) |
| Mycobacterium | NP_216767 | Bacteria | Camus J. C. et al., Microbiology |
| tuberculosis H37Rv | | | (Reading, Engl.) 148 (PT 10), 2967-2973 (2002) |
| Trypanosoma brucei | O97157 | Eukaryota | Zomer A. W. et al., Mol. Biochem. |
| brucei | | | Parasitol. 104 (1), 55-66 (1999) |
| Dictyostelium | O96759 | Eukaryota | De Vet E. C. et al., Biochem. |
| discoideum | | | Biophys. Res. Commun. 252 (3), 629-633 (1998) |
| Homo sapiens | O00116 | Eukaryota | De Vet E. C. et al, Biochim. |
| | | | Biophys. Acta 1346 (1), 25-29 (1997) |
| Cavia porcellus | P97275 | Eukaryota | De Vet E. C. et al, J. Biol. Chem. |
| | | | 272(2), 798-803 (1997) |
| Aeropyrum pernix K1 | BAA79263 | Archaea | Ren S. X. et al., Nature 422 (6934), |
| | | | 888-893 (2003) |
| Pan troglodytes | XP_515935 | Eukaryota | |
| Treponema denticola | NP_970755 | Bacteria | Fleischmann R. D. et al., |
| ATCC 35405 | | | J. Bacteriol. 1841, 5479-5490 (2004) |
| Leptospira interrogans | NP_712443 | Bacteria | Ren S. X. et al., Nature 422 (6934), |
| serovar Lai str. 56601 | | | 888-893 (2003) |
| Gallus gallus | XP_421987 | Eukaryota | |
| Sulfolobus solfataricus | AAK41753 | Archaea | She Q. et al., Proc. Natl. Acad. Sci. |
| P2 | | | U.S.A. 98 (14), 7835-7840 (2001) |
| Treponema denticola | AAS10636 | Bacteria | |
| ATCC 35405 | | | |
| Leptospira interrogans | AAN49461 | Bacteria | Ren S. X. et al., Nature 422 (6934), |
| serovar | | | 888-893 (2003) |
| Lai str. 56601 | | | |
| Archaeoglobus fulgidus | AAB90369 | Archaea | Klenk H. P. et al., Nature 390 |
| DSM 4304 | | | (6658), 364-370 (1997) |
| Pseudomonas | AAY92039 | Bacteria | Paulsen I. T. et al., Nature Biotech |
| fluorescens | | | 23 (7), 873-878 (2005) |
| Pf-5 | | | |
| Leishmania major | CAJ05782 | Eukaryota | |
| Dictyostelium | XP_637836 | Eukaryota | Eichinger L. et al., Nature 435 |
| discoideum | | | (7038), 43-57 (2005) |
| Homo sapiens | NP_055051 | Eukaryota | Liu D. et al., J. Lipid Res. 46 (4), |
| | | | 727-735 (2005) |
| Rattus norvegicus | NP_445802 | Eukaryota | |
| Dictyostelium | EAL64267 | Eukaryota | Eichinger L. et al., Nature 435 |
| discoideum | | | (7038), 43-57 (2005) |
| Caenorhabditis elegans | NP_497185 | Eukaryota | De Vet E. C. et al., Biochem. |
| | | | Biophys. Res. Commun. 242 (2), 277-281 (1998) |
| Mycobacterium | NP_337715 | Bacteria | Fleischmann R. D. et al., |
| tuberculosis CDC1551 | | | J. Bacteriol. 184 (19), 5479-5490 (2002) |
| Mycobacterium | NP_336781 | Bacteria | Fleischmann R. D. et al., |
| tuberculosis CDC1551 | | | J. Bacteriol. 184 (19), 5479-5490 (2002) |
| Caenorhabditis elegans | CAA05690 | Eukaryota | De Vet E. C. et al., Biochem. |
| | | | Biophys. Res. Commun. 242 (2), 277-281 (1998) |
| Caenorhabditis elegans | AAK68514 | Eukaryota | Fleischmann R. D. et al., |
| | | | J. Bacteriol. 1841, 5479-5490 (2004) |
| Homo sapiens | CAA70591 | Eukaryota | De Vet E. C et al., Biochim. |
| | | | Biophys. Acta 1346 (1), 25-29 (1997) |
| Mycobacterium | AAK47529 | Bacteria | Fleischmann R. D. et al., |
| tuberculosis CDC1551 | | | J. Bacteriol. 1841, 5479-5490 (2004) |
| Mycobacterium | AAK46595 | Bacteria | Fleischmann R. D. et al., |
| tuberculosis CDC1551 | | | J. Bacteriol. 1841, 5479-5490 (2004) |
| Mus musculus | BAC35474 | Eukaryota | |
| Mus musculus | BAC27229 | Eukaryota | |
| Suberites domuncula | CAD66418 | Eukaryota | |
| Leishmania major | AAP94009 | Eukaryota | Zufferey R. et al., J. Biol. Chem. |
| | | | 278 (45), 44708-44718 (2003) |
| Rattus norvegicus | AAG43235 | Eukaryota | |
| Trypanosoma brucei | AAD19697 | Eukaryota | Zomer A. W. et al., Mol. Biochem. |
| | | | Parasitol. 104 (1), 55-66 (1999) |
| Rattus norvegicus | CAB40909 | Eukaryota | |
| Dictyostelium | CAA09333 | Eukaryota | de Vet E. C., Biochem. Biophys. |
| discoideum | | | Res. Commun. 252 (3), 629-633 (1998) |
| Cavia sp. | CAA70060 | Eukaryota | de Vet E. C. et al, J. Biol. Chem. |
| | | | 272 (2), 798-803 (1997) |

The amino acid and nucleic acid sequences indicated in Table 1 and 2 can be found in the databases of the National Center for Biotechnology Information (NCBI) and the enzyme information system BRENDA of the Biochemical Institute of the University of Cologne.

The alkyldihydroxyacetone-phosphate synthase, which is highly preferred in some embodiments of the present invention, is that from Trypanosoma sp., Leishmania sp., Aeropyrum pernix, Sulfolobus solfataricus, Sulfolobus acidocaldarius or Archaeoglobus fulgidus. The corresponding nucleic acid and amino acid sequences of these enzyme catalysts are appended in SEQ. ID. Nos 1-12. The individual SEQ. IDs are assigned as follows:
SEQ: ID. No. 1: amino acid sequence of the ADAPS from Trypanosoma brucei
SEQ: ID. No. 2: nucleic acid sequence of the ADAPS from Trypanosoma brucei
SEQ: ID. No. 3: amino acid sequence of the ADAPS from Leishmania major
SEQ: ID. No. 4: nucleic acid sequence of the ADAPS from Leishmania major
SEQ: ID. No. 5: amino acid sequence of the ADAPS from Aeropyrum pernix
SEQ: ID. No. 6: nucleic acid sequence of the ADAPS from Aeropyrum pernix
SEQ: ID. No. 7: amino acid sequence of the ADAPS from Sulfolobus solfataricus
SEQ: ID. No. 8: nucleic acid sequence of the ADAPS from Sulfolobus solfataricus
SEQ: ID. No. 9: amino acid sequence of the ADAPS from Sulfolobus acidocaldarius
SEQ: ID. No. 10: nucleic acid sequence of the ADAPS from Sulfolobus acidocaldarius
SEQ: ID. No. 11: amino acid sequence of the ADAPS from Archaeoglobus fulgidus
SEQ: ID. No. 12: nucleic acid sequence of the ADAPS from Archaeoglobus fulgidus

In the amino acid sequences indicated in Tables 1 and 2 and the sequence ID numbers 1, 3, 5, 7, 9 and 11, and partial sequences thereof, one or more amino acids may have been deleted, added or replaced by other amino acids without a substantial reduction in the enzymatic effect of the polypeptide.

A functional variant means, in the context of the invention, an ADAPS comprising an amino acid sequence having a sequence homology of at least 30%, preferably of more than 60%, to one of the sequences referred in Table 1 and 2 or sequence ID numbers 1, 3, 5, 7, 9 and 11. A functional partial sequence additionally means ADAPS which comprises amino acid fragments composed of at least 50 amino acids, preferably composed of at least 100 amino acids, particularly preferably composed of at least 200 amino acids, but functional variants having deletions of up to 300 amino acids, preferably of up to 150 amino acids, particularly preferably of up to 50 amino acids, are also covered by the term functional partial sequence.

The ADAPS of the invention may additionally have post-translational modifications such as, for example, glycosylations or phosphorylations.

The present invention further relates to the use of the translation products of nucleic acids having one of the sequences referred to in Table 1 or 2 or sequence ID numbers 2, 4, 6, 8, 10 or 12 for preparing ethers. The invention additionally relates to the use of translation products of partial sequences or nucleic acid sequences which, as a consequence of the degeneracy of the genetic code, have a different nucleic acid sequence but code for the same polypeptide according to one of the amino acid sequences referred to in Table 1 or 2 or the sequence ID numbers 1, 3, 5, 7, 9 and 11, or for an analog, allele, derivative or a partial sequence thereof in which one or more amino acids have been deleted, added or replaced by other amino acids without a substantial reduction in the enzymatic effect of the polypeptide, for preparing ethers.

The invention additionally includes translation products of allelic or functional variants of one of the nucleic acid sequences referred to in Table 1 or 2 or sequence ID numbers 2, 4, 6, 8, 10 or 12, having a homology of more than 50%, preferably of more than 75%, particularly preferably of more than 90%, or their partial sequences composed of at least 150 nucleotides, preferably composed of at least 300 nucleotides, particularly preferably composed of at least 600 nucleotides, or fragments which are complementary to those nucleic acid sequences hybridizing with a coding sequence referred to in Table 1 or 2 or sequence ID numbers 2, 4, 6, 8, 10 or 12, or an allelic or functional variant or one of their partial sequences under stringent conditions.

It is possible to employ according to the invention whole cells, resting cells, immobilized cells, purified enzymes or cell extracts which comprise the corresponding enzymes, or mixtures thereof. The enzymes may be used according to the invention in whole-cell systems, in free form or immobilized on suitable supports.

In the process of the invention, the reactants are mixed and, optionally, a nonaqueous solvent can be added. The appropriate enzyme, a cell extract or whole cells which comprise the. desired enzyme are added, and the reaction mixture is maintained at the temperature optimal for the enzyme used, normally 15° C. to 100° C., preferably 20° C. to 70° C. The reaction is monitored by standard analytical methods, for example by gas chromatography.

The following examples are provided to illustrate some aspects of the present invention.

### EXAMPLE 1

### Preparation of Recombinant ADAPS from Trypanosoma brucei

500 mL of ampicillin-containing (100 mg/L) Luria Bertani medium was inoculated with an overnight culture of E. coli BL21l(DE3)pLysS which harbors the plasmid pET15b in which the ADAPS gene (SEQ ID No. 2) was encoded. Cultivation took place at 37° C. and, when an OD₆₀₀ of 0.5 was reached, ADAPS production was induced by adding IPTG (0.4 mM). 4.5 hours after induction, the cells were removed by centrifugation (4000 g, 4° C., 15 min). The resulting pellet was resuspended and then recentrifuged twice in 20 ml of ice-cold potassium phosphate buffer (50 mM, pH 7.5) each time. Finally, the cells were disrupted by sonication with ultrasound (50% power, 50% pulse) on ice for 10 minutes. The cell envelopes were removed by centrifugation, and the supernatant was lyophilized for use in the organic synthesis. The protein content was determined by the Bradford method using bovine serum albumin as a reference. Protein content of the crude cell extract: about 5 mg/mL (total protein content: 100 mg).

### EXAMPLE 2

### ADAPS Purified Via His Tag

The ADAPS was further purified by metal ion chromatography using the Talons™ method in accordance with the manufacturer's protocol. This afforded 3.5 mg of purified ADAPS from the above culture batch.

### EXAMPLE 3

### Analysis of the ADAPS Reaction

Samples of the reaction mixture were analyzed by gas chromatographic analysis (Hewlett Packard GC HP 5890 Serial II plus) with a flame ionization detector and optima 17 TG column (MACHEREY-NAGEL GmbH & Co. KG, Düren). The following temperature program was used:

Injector temperature: 245° C.

Detector temperature: 245° C.

| Oven temperature [° C.] | Time [min] |
|---|---|
| 160 | 0 |
| 160 | 0.5 |
| 190 | 2 |
| 260 | 3.4 |
| 260 | 5.4 |

In ADAPS-catalyzed reactions with phosphorylated compounds, only alcohol and fatty acid were detected, and with all non-phosphorylated compounds all the substrates and products were quantified with this method.

Samples of the reaction mixture (100 μL) were, in some instances, freeze dried (aqueous system) or concentrated with nitrogen (evaporated, solvent system). Chloroform (20 μL) and 2 μL of an internal standard (10-20 mmol) were then added to the reaction mixture. The mixture was analyzed in a gas chromatograph.

### EXAMPLE 4

### Enzymatic Conversion of Palmitoyldihydroxyacetone (PDHA)

90 μL palmitoyldihydroxyacetone (PDHA) and 90 μL n-octadecanol were dissolved in 800 μL of potassium phosphate buffer (50 mM, pH 7.5, 50 mM NaF, 0.1% [w/v] Triton X-100). 200 μL of a protein solution from Example 1 was added, and the reaction was carried out in a 1 mL reaction vessel with shaking (1000 rpm) at 37° C. Samples with a volume of 100 μL were taken at intervals and analyzed as in Example 3. After approximately 4 hours, an equilibrium was set up at about 45% conversion. The sole figure of the present application illustrates the progress of an ADAPS-catalyzed reaction for various materials as in this example

### EXAMPLE 5

### Enzymatic Preparation of 1-Octyldecyldihydroxyacetone in Potassium Phosphate Buffer

50 mg (0.15 mmol) of palmitoyldihydroxyacetone (PDHA) and 50 mg (0.19 mmol) of n-octadecanol were dissolved in potassium phosphate buffer, 50 mM NaF, 0.1% [w/v] Triton X-100 (100 mL). 20 mL of a protein solution from Example 1 were added, and the reaction was carried out in a 1000 mL flask stirred with a magnetic stirrer at 37° C. The reaction was stopped by centrifugation (4000 g, 15 min, 4° C.) in order to remove the enzyme. The aqueous phase was removed by freeze drying, and the crude product was purified by column chromatography on silica gel (chloroform:methanol, 2:1). Yield: 8 mg of 1-octadecyldihydroxyacetone.

Analytical data: ¹H-NMR (chloroform-d, D=99.8) δ in ppm: CH₃ 0.89 3H; CH₂ 1.27 28H; CH₂ 1.31 2H; CH₂ 1.59 2H; OH 2.36 2H; CH₂ 3.56 2H; CH₂ 4.25 2H, ¹³C-NMR (chloroform-d, D=99.8): CH₃ 14.43; CH₂ 23.35; CH₂ 30.07; CH₂ 30.28; CH₂ 30.42; CH₂ 32.65; CH₂ 67.62; CH₂ 70.01; C═O 173.11.

### EXAMPLE 6

### Enzymatic Preparation of 1 -Tetradecyldihydroxyacetone in Potassium Phosphate Buffer

On reaction of 100 mg of palmitoyldihydroxyacetone (PDHA) with myristyl alcohol in analogy to Example 5, it was possible to isolate 12 mg of the desired product 1-tetradecyldihydroxyacetone.

Analytical data: ¹H-NMR (chloroform-d, D=99.8) δ in ppm: CH₃ 1.0 3H; CH₂ 1.31 22H; CH₂ 1.5 2H; OH 2.1 1H; CH₂ 3.4 2H; CH₂ 4.5 4H, ¹³C-NMR (chloroform-d, D=99.8): CH₃ 14.1; CH₂ 22.7; CH₂ 28.1; CH₂ 30.4; CH₂ 70.1; CH₂ 78.1; C═O 202.1.

### EXAMPLE 7

### Enzymatic Preparation of 1 -Hexadecyldihydroxyacetone in Potassium Phosphate Buffer

On reaction of 100 mg of palmitoyldihydroxyacetone (PDHA) with hexadecanol in analogy to Example 5, it was possible to isolate 16 mg of the desired product 1-hexadecyldihydroxyacetone.

Analytical data: ¹H-NMR (chloroform-d, D=99.8) δ in ppm: CH₃ 1.1 3H; CH₂ 1.3 24H; CH₂ 1.4 2H; CH₂ 1.5 2H; OH 2.3 1H; CH₂ 3.4 2H; CH₂ 4.6 4H, ¹³C-NMR (chloroform-d, D=99.8): CH₃ 13.9; CH₂ 23.0; CH₂ 27.1; CH₂ 30.0; CH₂ 70.3; CH₂ 77.1; C═O 0 207.0.

### EXAMPLE 8

### Enzymatic Preparation of 1 -Alkylglycerol Ethers in Potassium Phosphate Buffer

90 μL of 1-palmitoylglycerol or 1-lauroylglycerol and 90 μL n-tetradecanol, n-hexadecanol or n-octadecanol were dissolved in 800 μL of potassium phosphate buffer (50 mM, pH 7.5, 50 mM NaF, 0.1% [w/v] Triton X-100). 200 μL of a protein solution from Example 1 was added, and the reaction was carried out in a 1 mL reaction vessel while shaking (1000 rpm) at 37° C. Samples with a volume of 100 μL were taken at intervals and analyzed as in Example 3. The following conversions were determined after about 22 hours:

| Glyceride | Alcohol | Conversion 22 h [%] |
|---|---|---|
| 1-Palmitoylglycerol | n-Tetradecanol | 9 |
| 1-Palmitoylglycerol | n-Hexadecanol | 5 |
| 1-Lauroylglycerol | n-Tetradecanol | 7 |
| 1-Lauroylglycerol | n-Hexadecanol | 13 |
| 1-Lauroylglycerol | n-Octadecanol | 10 |

### EXAMPLE 9

### Enzymatic Preparation of 1-Octadecyldihydroxyacetone in n-Hexane

50 mg (0.12 mmol) of PDHA and 50 mg (0.19 mmol) of n-octadecanol were dissolved in n-hexane. 1 g of lyophilized cell extract from Example 1 was added, and the reaction was carried out in a 250 mL flask stirred with a magnetic stirrer at 37° C. The reaction was stopped by centrifugation (4000 g, 15 min, 4° C.) in order to remove the enzyme. The organic phase was freed of solvent, and the crude product was purified by column chromatography on silica gel (chloroform:methanol, 2:1). Yield: 7.5 mg of 1-octadecyldihydroxyacetone.

Analytic data: ¹H-NMR (chloroforn-d, D=99.8), δ in ppm: CH₃ 1.0 3H; CH₂ 1.3 28H; CH₂ 1.4 2H; CH₂ 1.3 2H; OH 2.1 1H; CH₂ 4.4 4H; ¹³C-NMR (chloroform-d, D=99.8): CH₃ 14.0; CH₂ 23.0; CH₂ 30.0; CH₂ 30.7; CH₂ 33.0; CH₂ 68.2; CH₂ 80.1; CH₂ 70.9; C═O 198.1

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A process for preparing ethers of the general formula (I)
R—O—-R¹ (I)
in which R is a hydrocarbon radical of an alcohol and has 1 to 30 C atoms, which comprises reacting one or more alcohols of the general formula (II)
R—OH (II)
in which R is as defined above, in the presence of at least one enzyme, with a compound of the general formula (III),
R¹—O—R² (III)
in which
R¹ is a linear or branched, alkyl or alkenyl group having 2 to 30 carbon atoms, and
R² is an acyl radical of an acid and has 2 to 30 carbon atoms, with the proviso that when R¹ is a 1-dihydroxyacetone phosphate, R² is not an acyl radical of a naturally occurring fatty acid having 8 to 30 carbon atoms.

2. The process as claimed in claim 1, wherein the reaction is carried out in a nonaqueous reaction system with a water content of less than 30%.

3. The process as claimed in claim 1, wherein the alcohol residue R has 2 to 30 carbon atoms.

4. The process as claimed in claim 1, wherein the radical R¹ is selected from the group consisting of substituted or unsubstituted dihydroxyacetone, 1,2-propylene glycol, 1,3-propylene glycol, neopentyl glycol, trimethylolpropane, pentaerythritol, sorbitol, 1,6-hexanediol, 1,2-pentanediol, glycerol, diglycerol, triglycerol, polyglycerol, diethylene glycol, triethylene glycol and polyethylene glycol.

5. The process as claimed in claim 4, wherein free hydroxyl groups of the radical R¹ are esterified with an inorganic acid, an acyl radical, or an additional alcohol.

6. The process as claimed in claim 1, wherein R² is selected from the group consisting of substituted or unsubstituted acetic acid, propanoic acid, butanoic acid, pentanoic acid, chloroacetic acid, trifluoroacetic acid, substituted and unsubstituted acyl radicals of monobasic fatty acids based on natural vegetable or animal oils having 6 to 30 carbon atoms, and mixtures thereof.

7. The process as claimed in claim 1, wherein said at least one enzyme is a cell extract comprising enzymes or mixtures thereof.

8. The process as claimed in claim 1, wherein said at least one enzyme is from the class of transferases that transfer alkyl groups (EC 2.5.x.y).

9. The process as claimed in claim 8, wherein at least one alkyldihydroxyacetone-phosphate synthase (ADAPS, EC 2.5.1.26) or an allelic or functional variant thereof or a functional partial sequence thereof is employed.

10. The process as claimed in claim 9, wherein an alkyldihydroxyacetone-phosphate synthase having one of the amino acid sequences as referred to in Table 1 or 2 or one of the SEQ ID No. 1, 3, 5, 7, 9 or 11 is employed.

11. The process as claimed in claim 10, wherein one or more amino acids have been deleted, added or replaced by other amino acids without substantially reducing the enzymatic effect of the polypeptide.

12. The process as claimed in claim 1, wherein the enzyme employed is a translation product of a nucleic acid sequence as referred to in Table 1 or 2, or one of the SEQ ID No. 2, 4, 6, 8, 10 or 12, or an allelic or functional variant thereof or their partial sequences or fragments which are complementary to those nucleic acid sequences hybridizing with coding nucleic acids.


SEQUENCE LISTING (12 sequences)

SEQ ID NO: 1 - protein, 613 aa, Trypanosoma brucei <s300> Anna W.M. Zomer, Paul A.M. Michels, Fred R. Opperdoes Molecular characterisation of Trypanosoma brucei alkyl dihydroxyacetone-phosphate synthase Molecular and Biochemical Parasitology 104 55 - 66 1999 (1)..(613)
MDKRMITDAFEEIKWNGWGDTGVCIKYDEARQLPIHTNGKPMKHLLKFMKDDVLKVKGEFKIKPTPGLTKEEAIKRLPPPVVKQPFVDELRQVLSKDQIRLDAYARLTHIFGKNYRDLWRVRRGMIDRPPDAVILPNNHDDCVKIMELAQKHNVVVVPFGGGTNVTGGVEPNPFETRRMVISIDMRRMGRMLHIDTESGTAVFEVGVLGPDIDEQLSRYGFMMGHDPDSYAYSTLGGWIAARGSGAMSNKYGDIENMILAMRVVTPVGVVETPLTSRPCGVDLNAMFVGSEGAFGLVTEAVVKIERLPEVKRYEGWLFPSFEVAFTAFHTCTRKGIHPCTMRLYDEDDTRLSFAASTDSGLVSTFFSKCFKKYIATVKGWNLSKISLVVVGFEGTKAQTNCQRSELVGVFQAFGATCLGTKPGNTWQEKKYDLPYLRDFALAHNFWADVFETSVLYTDAIHCWRAVKKSFAEVMAENGKNAWIGCHTAHQYRFGCCLYFTFIGGQADENDLKIFLQVKKRAMEVMLQHRGNLTHHHGIGYEHVPWMKRYNGEGGLDAIMKFKKALDPKNICNPGKLLPSPPSEKETPKATQARQNREMMFDKMGIPGALQAHL

SEQ ID NO: 2 - DNA, 1842 bp, Trypanosoma brucei <s300> Anna W.M. Zomer, Paul A.M. Michels, Fred R. Opperdoes Molecular characterisation of Trypanosoma brucei alkyl dihydroxyacetone-phosphate synthase Molecular and Biochemical Parasitology 104 55 - 66 1999 (1)..(1842)
atggataagagaatgattactgatgcatttgaagaaatcaaatggaatggctggggggacactggcgtgtgtatcaaatacgatgaggcgaggcaactccccatacacacaaatggaaagccgatgaagcatcttctgaagttcatgaaggacgatgtcctcaaggtgaagggagagtttaaaattaagcccacacccgggctaacaaaagaggaggccattaaacggctcccaccacctgtagtgaaacaaccattcgtggatgagttgcgtcaggttttgtcaaaagatcagatccgtctcgacgcatatgcccgcctgacgcacatctttggtaaaaattatcgcgacctttggcgtgtgaggcgtggtatgatcgaccgcccacccgatgcggtcattctccccaacaaccacgatgattgcgtaaagattatggaattggcacaaaaacacaacgttgttgttgtgccattcggtggtggcacaaatgtgacaggtggcgtggagccgaatccctttgagacgcgacgtatggttatctccattgacatgcgacggatggggcgaatgcttcatatagacacagaatctggtaccgctgtgtttgaagtgggagtgcttggcccagatatagatgagcaactgagccgctacggtttcatgatgggtcatgaccccgatagttatgcatattcgactcttggtggctggattgctgcgcgaggcagcggtgccatgtcgaataaatatggagatattgaaaacatgattctcgccatgcgggtggtgacacctgttggcgtggtggaaacaccccttacctcgcgaccctgcggtgtcgacttgaacgccatgtttgttgggtccgagggtgcctttggacttgttacagaggctgttgttaagattgaacgcctaccagaggtgaagcgttacgaaggctggctctttccctcatttgaagttgcttttactgctttccatacatgcacacgcaaggggatccacccttgcacaatgaggttatacgacgaagacgacacacggctcagttttgcagcaagtacagactccgggttggttagtacattcttcagcaagtgctttaagaaatacatcgcaactgtgaagggatggaacctcagcaaaatatcacttgtggttgtcgggtttgaaggaaccaaagcccaaacgaactgtcagcggagtgaattggttggcgtcttccaagccttcggtgccacatgcttgggaaccaagccgggtaacacgtggcaagaaaagaaatacgaccttccgtaccttcgcgatttcgctctggctcacaatttttgggcggacgtatttgagacgagtgtcctctacacggacgctattcactgctggagagcagtcaagaaatcgtttgcggaagtgatggccgagaatggtaagaacgcatggataggctgccacactgcacatcagtaccgttttggttgctgtttgtatttcacctttattggcgggcaggccgacgaaaatgacctcaagatctttttgcaagtaaaaaaaagggcgatggaagtcatgttacagcacagaggaaacctaactcaccaccatggaataggttatgagcacgtcccatggatgaagcgttacaatggtgagggtgggttagacgcaattatgaagttcaagaaagcgcttgatccaaagaacatatgtaacccgggaaaactgctcccctcacccccttcggagaaggaaacaccaaaggccacacaggcccgtcagaatcgtgaaatgatgtttgacaaaatgggcattcccggggctcttcaggctcacctctga

SEQ ID NO: 3 - protein, 620 aa, Leishmania major
MPADEAQAPTQTEVYDRLKWNGWGVEDVQMQIDDENPLWVRHVDGKPIKNLLEFLYQEISGVGPKEIPPLSPSIPLEKALAKLNKANINEAFMKDISAVLEKSQIRPDGKSRLCHIAGKNYRDLWRVRKGMVEHAPDCILLPNSHKDCVEIMRLAHKHNVVVIPFGGGTNVTGGVEADPFERARMIISVDMRRMNRMISIDRESRLATFETGVLGPDLDAQLFRHGFMLGHDPDSYIYSTLGGWIGARGSGAMSNQYGDIEDMVVAVKVATPTGIIETPTTSRTCGVDLNGLFIGSEGSFGIITEATIKLETIPETKLYEGYLFPTFEAGFSAFYTCTAKGIHPCTMRLYDEDDFRMSMAMSTTKHSFFQRLVSMGVKSFLERYRGWNLRRISLVIVGFEGTPDRVKFQRSETAAVFKQYGGVGVGRSAGATWQDKKYDLPYIRDFALSLSHWADVFETSVLYSQAIPCWRAVKAAVRQVWKEHGHRGWIGCHTAHQYKYGCCLYFTFASAQKDDMDMKIFLAIKKRATEAMLAHTGNLTHHHGIGYEHVPWMSRYMGPDALDLLFAVKQKVDPKNICNPGKLLPSPPRENESAAALKARQQRELMFDKMGVPGAVASHI

SEQ ID NO: 4 - DNA, 1866 bp, Leishmania major
atgcccgctgacgaggcgcaggcgcccacccagacggaggtgtatgatcgcctcaagtggaatgggtggggtgtcgaggatgtccagatgcagatcgacgacgagaacccgctctgggtacgccacgtggatggcaagccgatcaaaaacttgcttgagttcctctaccaggagatcagcggcggagtaggtccaaaggagatcccgccgctgtcgccgagcatcccgctagagaaggcacttgcgaaacttaacaaggcgaacatcaacgaggccttcatgaaggacatctccgcggtgctggagaagagccaaattcgccctgacggcaagagtcgtctgtgccacattgcgggcaaaaactaccgcgacctgtggcgtgtgcgcaagggcatggtcgaacacgctccagactgcattctgctgccgaatagccacaaggactgtgtcgagattatgaggctggcgcacaagcacaacgtggtcgttattccttttggtggcggcaccaacgtgacgggtggcgtcgaggcagaccccttcgagcgcgcccgcatgatcatctccgtggacatgcgccgcatgaaccgcatgatatcgattgaccgcgagtcgcgtctggccacctttgaaaccggtgtgcttggccctgacctggatgcgcagttgttccgccacggcttcatgttgggccacgaccccgacagctacatctactcaaccctcggcggatggattggtgcacgcggcagcggggccatgtcgaaccagtacggcgacattgaagacatggtggtggcggtgaaggtggcaacgccgacaggcatcatcgaaaccccaaccacgtcccgcacgtgcggcgtggacctgaacgggctcttcatcggctccgaaggctccttcggcatcattacggaggccaccatcaagctggaaaccatcccggagacgaagctatacgaagggtacctgttccccaccttcgaggctggtttcagcgccttctacacatgcaccgccaaaggaatccatccgtgcacgatgcgcctgtacgacgaggacgactttcgtatgagtatggcgatgagcaccaccaagcacagcttcttccagcgcttggtgagtatgggtgtcaagtccttccttgagagataccgcggctggaacctgcgccgcataagcctagtgatcgtcggctttgagggaacgccggatcgcgtcaagttccagcgaagcgagacggctgcagtcttcaagcagtacggtggcgtcggtgtcggccgcagcgcgggtgctacgtggcaggataagaagtacgacctgccgtacatacgtgacttcgccctgtctctctcgcactgggcggacgtcttcgagacgagcgttctctactcgcaggcgatcccgtgctggcgtgcagtgaaggccgctgtgcggcaggtgtggaaggaacacgggcaccgcggctggattggctgtcacacggcccaccagtacaagtacggctgctgcctctacttcaccttcgcaagcgcgcaaaaggacgacatggacatgaagatattcctcgccatcaagaagcgtgcgacggaggcgatgctggcgcacactggaaacctcacccaccaccacggcatcggctatgagcacgtgccgtggatgagccgctacatggggcctgatgcgctggacctcctctttgctgtgaagcagaaagtggacccaaagaacatctgtaaccctggaaagttgctgccgtcgcctccacgggagaacgagagcgccgcagcgctgaaggcacgccagcagagggagctgatgttcgacaagatgggcgtgccaggagccgtcgcgtcgcatatttaa

SEQ ID NO: 5 - protein, 430 aa, Aeropyrum pernix <s300> NP_147137 2005-04-08 (1)..(430)
MWPLWPLLVEKGFRPRAYVAKPRSRGAVERIVEAAAEAGACLVPRGGGSNVVGASPPLGCCLILDLRMLDKILWFSGEDLVVHVEAGAVVSKVEEWLNRRGYTLGYHPQSQSLATVGGSIAMLGSGALAPGLGNIEDMVLWLETVIPGLGTVTLGSRNSPRGWEGPGVKHLFIGSEGSLGVVVSAGLRVRPLPEFTGALAYRLPGFREGLEAARRLTLWRGARLLRLLDPSEASMLYGVDGAVLMVEVEAPDRGLLEAMEGYVEKVASASGGSRVEGVYEKWARARYMYDEHVRQLWSAGLWVDTIDTAAPWSRVEDLNRRLLEDLAGIPGVVAVMSHAGHFYSGGASLYHTVVMERRLDTYWRVWSRVAEAVRQLGASITHQHGWGLLRKPYLGFLGGNYRVFCRVKNTLDPGNVLNPNGISSRCSRVG

SEQ ID NO: 6 - DNA, 1293 bp, Aeropyrum pernix <s300> NP_147137 2005-04-08 (1)..(1293)
atgtggccgctctggccgttgctggtggagaaggggttcaggcctcgtgcgtatgtggcgaagccgcggagccgaggggctgttgagaggattgtggaggcggctgcggaggctggagcatgcctcgtgcctaggggaggcggctctaacgtcgtgggcgcctcacccccgctcggctgctgcctcatacttgacctgaggatgctggacaagatattgtggttcagcggtgaggacctcgttgtccacgtcgaggcgggggctgttgtctcgaaggttgaggagtggctgaacaggcggggatacacactgggctaccacccccagtcccagagccttgccactgtaggaggctccatagccatgctaggctcgggcgccctagcaccggggctgggtaacatagaagacatggtactctggctggaaacagttataccgggtctcggaaccgtcacgctaggctcccgcaacagcccccgtgggtgggaggggccgggggtcaagcacctattcatagggtctgagggctcactgggcgtcgtcgtatcggcgggcctcagggtcaggccgctgcccgagtttacaggcgccctggcctacaggctgcccggcttccgcgagggtctagaggctgcgagacggctcacactatggaggggcgccaggcttctgaggcttctagaccccagtgaggctagcatgctctacggcgtcgacggcgccgtgctcatggtggaggttgaggcgccagataggggtttgctggaggctatggaaggctatgttgaaaaggtcgcctccgccagcggaggttcaagggttgagggtgtctacgagaagtgggctagggctaggtacatgtacgacgagcacgttaggcagctctggagcgccggtctatgggtcgacactatagacacggcagccccctggagcagggttgaggacctcaacaggaggctcctggaggacctcgcggggattccaggcgttgtagctgttatgagccacgcgggccacttctacagcgggggggcgagcctataccacaccgtcgttatggagaggaggcttgacacgtactggagggtctggtctagagtggcagaggctgttagacaactgggcgcctcgataacccaccagcacggctggggcctgctcaggaagccctacctagggtttttaggcgggaactacagggtgttctgccgcgtcaaaaacaccctggaccccgggaacgtcctcaaccccaacggcatctcctcgaggtgcagcagggttggatag

SEQ ID NO: 7 - protein, 451 aa, Sulfolobus solfataricus <s300> NP_342963 2005-03-08 (1)..(451)
MQEFINEIQRNFSNCVVEDLDKYKRDWTPLLILEEHLGKNLGKPLLVLSPSTTEEVSNIVKLANKYYVCIVPYGGGSSVVGGSYHNNCVILDLSKLNRIIEFNEEDLSVTVEVGIKIKDLEEWLNKRGYTLDYHPQSFYLATIGGAIAHKGSGSHSSSNIENLILWLEVILPNGKAVKVGSSIRTSMSPDLVRLFIGSEGTLGVITKAKLRIFPLAPYHTDLSFLFPNFHNAIKFTREYVLRTSPPHRIVVHDKESSEMMLNIPYNIALIRVRGYDEDLVNVNEKIIRQLAMRHNANEGDRSLIRSWREVFARKYEEQMTKIISQGLWNDTLDLGATWSAMPKLYDKLKESLNSMKGIKNVLSRVTHVYTNGASLYNVVVMEQDLELLKRVWETAARVIMQLGGTISHHHGVGILKKNWVKEELGEQYDLLLLIKRMIDSNNIMNPGKVFS

SEQ ID NO: 8 - DNA, 1356 bp, Sulfolobus solfataricus <s300> NP_342963 2005-03-08 (1)..(1356)
atgcaggagtttataaatgaaattcagaggaatttttcaaattgcgtagtggaagacttagataagtataaaagggactggacaccactcttaattctagaagaacatttaggaaaaaatttaggaaaaccgttgttagttctctctccatcaactaccgaggaggtttcaaatattgttaagttggctaataagtattatgtctgcatcgttccttacggaggaggttctagcgtagtgggtgggtcttatcataataattgcgtaatccttgatttatctaaactaaacagaataatagagttcaatgaggaagacctaagcgtaacagttgaggtaggaataaaaattaaggatcttgaagaatggttaaataaaagaggatatactctggattatcatcctcaatccttttatctagctaccataggtggggctatagctcataaaggatctggatcacatagtagtagtaacatagagaatttaatattgtggttagaagtgattttgcctaacggtaaagcagttaaagtcggtagttccataagaacttccatgagtccagatcttgtaaggttatttataggctccgagggaactttaggagtaataactaaggctaaactgagaatattccctcttgcaccatatcatactgacctttcctttctctttcctaattttcataatgcaataaagtttactagggaatatgttttgaggacctctcctccacataggatagtagttcacgataaggaaagcagtgaaatgatgttgaatatcccttataatattgcattaataagagttaggggatatgacgaagatttagttaacgtgaatgagaagataattagacagcttgctatgagacataacgcaaatgagggcgacagaagtctgataagaagctggagagaggtctttgctagaaagtatgaagagcaaatgactaagataatttcacaaggtttatggaatgacacattagatttaggtgctacttggagtgcgatgcctaagctttatgataagctaaaggaatccttaaactccatgaaaggtattaaaaatgttctgtccagagtgactcatgtctatactaacggtgcctcactatataacgtggtggttatggaacaagaccttgagttattaaaaagagtttgggagactgctgctagagttataatgcaattaggtggtacaatttcccaccatcatggagttggcattttaaagaaaaattgggttaaggaagaattaggcgagcaatacgacttattgcttctaattaaaagaatgattgatagtaataacataatgaatccaggtaaagtgttttcataa

SEQ ID NO: 9 - protein, 415 aa, Sulfolobus acidocaldarius <s300> YP_256483. 2005-07-08 (1)..(415)
MEAETITKTFEKEFGDKKFITNDRVLEEESKAPYLVSPVLSKMGKKVIGVVMADDEEDIFNAVRLCDTHHIPLLARGAGTSTIGQVLPISPSTIVLDINRMREVIESDDSWVRITPSVKVFKALQYLRKRGKELRVYPSSFYISTLGGYIAGGDVGIGSFQYGYHFDNGGINSVRVVGPTGKYELRGEDTLGVAQAAGTTGIISSAELATVDYEDWKDQLVTFNSVEEVTKYLVRVRDYKSKIRRITIEDKETLSSVAKDRLDKVGEWNVIVASTLSLGEEVNMRFLDELAFAAIYVTMSRLTSFKDYFYEVRLLPLESFSRVVKQVKMALGSSVLVHGDVMTLRGETIVYTVFMSDKGNFPVIDSIMTREGIPFEIHSIEVNYRVDEESRLNLMKKLKRTIDPNNILNPGKLLL

SEQ ID NO: 10 - DNA, 1248 bp, Sulfolobus acidocaldarius <s300> YP_256483. 2005-07-08 (1)..(1248)
atggaagctgagaccattacaaaaacctttgagaaggagttcggcgacaagaagttcataacgaacgacagagtcctagaggaagagtccaaggctccataccttgtctccccagtcctatctaaaatgggcaaaaaagtgataggggtcgtcatggcagatgacgaggaggacatatttaacgctgttaggttgtgtgacacacaccacataccactgctagccaggggagccggtaccagtactataggacaggtcttacctatttctccctcaaccattgtcttagacatcaacagaatgagggaagtaatagagtcagacgactcctgggtgaggataacccccagtgtgaaagtgtttaaggctcttcagtacctgagaaagaggggtaaagagctaagggtctacccaagcagcttctacatatccaccctggggggatatatagcagggggagacgtagggataggctcatttcagtacggctaccacttcgacaacggagggataaactccgtcagagtcgttggacccactgggaagtatgagctaaggggtgaggacacactaggggtagcccaagcagccgggactacgggtataatctcctcagcggagttggcaacagtggactatgaggactggaaggaccaactagtcacgttcaacagcgtagaggaagtgacaaagtacttggtgagagtaagagactacaagtccaagataaggaggatcacaatagaggacaaggagacactctcgtcagtagccaaggacaggttagataaggtaggtgagtggaacgtaatagttgctagcactctgagcctaggagaggaggtcaacatgaggttcctggacgaacttgcctttgctgcaatttacgtcacaatgagcaggttgaccagctttaaggactacttctacgaggtaaggctcttacccttagagagcttctcaagggtagtaaagcaggtgaagatggctctaggcagtagtgtattagtccacggggacgttatgaccctgaggggagagacaatagtctacacagtgttcatgtcagataagggcaatttcccagtgattgacagcataatgacaagggaggggataccctttgaaattcactcaatagaggtgaactacagggtggacgaggagtcaaggttaaacttaatgaagaagttgaagaggaccattgaccccaacaacatactcaacccggggaagttgttactatga

SEQ ID NO: 11 - protein, 447 aa, Archaeoglobus fulgidus <s300> NP_069702 2005-04-08 (1)..(447)
MIAIEKALEEIRAAGIAVSTNMEDRIAYCKDYNPLHLPKLLRREFSAMPDAVVFPKSTEEVSVILKVANTYGIPVYVFGGGSGVIDAATPYEGGIALSTLSLDQIEIDEENMVVRAGAGVVGGRLEKILNHRGFTLRHSPQSLYCSTVGGWVATAASGQFSTGYGNIENLLVSLTAVLPDGEVVTERATPRRAGPDLKKIFVGSEGLLGVVTEAVMKIFPLPEEFLTLSVEYDGIGEAVRDAKALMQLRPALMRIFDDEESLRYFDSESFTLIAVFEGRAASAKLKEARNLVRGRLVEGRAEKWLEKRFDVSDISRIVPLGFIFDTIEVACFWKDAERVYSEVLKAIRSVEGTVTASAHASHFYESGLCFYFTFAGLPADIESYYFEVWRRAIEASLKNGGNLTHHHGVGRLRKRWLSAEIGGYYPLLRDLKSVLDRRNILNRGVML

SEQ ID NO: 12 - DNA, 1344 bp, Archaeoglobus fulgidus <s300> NP_069702 2005-04-08 (1)..(1344)
gtgatagccatcgaaaaagctcttgaggaaatcagggctgctgggatagcggtctccacaaacatggaggacagaattgcctactgcaaggactacaatccccttcaccttcccaagctgctcaggcgagagttttctgcaatgcctgacgctgttgttttcccgaagagcactgaggaggtttcagtcattctcaaggtcgcaaacacctacggcatacctgtgtacgttttcggcggtgggagcggggttatagatgcagcaactccctacgaggggggaatagctctctccacgctctccctcgaccagatagagattgatgaggagaacatggttgttagagcgggagctggggttgttgggggaaggctggagaagattctgaaccatcggggcttcacgctccgccactctccccagagcctctactgctcgaccgttggcggctgggttgctacggcagcgagcgggcagttctcaactggctacggcaacatcgagaaccttcttgtctccctaacggctgttctcccggatggtgaagtggttacggagagggcaactcccagaagggctgggcccgatttaaagaaaatttttgtgggaagcgaggggctgctcggtgttgttaccgaagcggtaatgaagatttttccattgccggaagagtttctaacgctctcagtcgaatacgatggtattggtgaggctgtgagggatgcaaaggctttgatgcagctaagaccagctctcatgagaattttcgacgatgaggagagtctgaggtacttcgactcggaaagcttcactctgatagcggttttcgagggaagggctgcgtctgcaaagctgaaggaggccagaaatctcgttaggggaaggttggttgagggaagggcggagaagtggcttgagaagaggttcgacgtttcggatataagccgaatcgtccccctcggcttcatcttcgacaccatagaggttgcgtgcttctggaaggatgcggagagagtttactcagaggttttgaaggccataaggagcgttgagggaacagtcacagcctcagcccacgcatcgcacttctacgagagcggtttgtgcttctacttcacctttgccggtttgccggcagacatagagagctactactttgaagtatggaggagggccatcgaggcttcgctgaagaacggtgggaatctgacgcaccaccacggcgtggggaggttgaggaagaggtggctttcggcagagattgggggatactatcctctgctgagagacctgaagagcgttctggacaggaggaacatcctcaacaggggggtgatgctgtga